(19) United States
(12) Patent Application Publication
(10) Pub. No.: US 2016/0354105 A1
(43) Pub. Date: Dec. 8, 2016

(54) Surgical Blade with Viewing Aperture

(71) Applicant: SEGWAY ORTHOPAEDICS, INC., Carlsbad, CA (US)

(72) Inventor: Stuart SEYMOUR, Carlsbad, CA (US)

(21) Appl. No.: 15/104,464

(22) PCT Filed: Nov. 26, 2014
(86) PCT No.: PCT/US2014/067743

§ 371 Date: Jun. 14, 2016

Related U.S. Application Data
(60) Provisional application No. 61/909,294, filed on Nov. 26, 2013.

(51) Int. Cl. A61B 17/32; A61B 90/00

(52) U.S. Cl. CPC A61B 17/320036; A61B 17/320016; A61B 90/08; A61B 2017/320052; A61B 2090/08021

(57) ABSTRACT

A surgical device configured for arthroscopic surgeries which require an imaging component communicating an image to a display to guide the user. The device provides a surgical blade having a viewing aperture formed therein. The aperture provides a line of sight view of the area opposite the side on which the imaging component is positioned. The blade may be combined with a guide to both protect from accidental cutting during maneuvering of the device within tissues of a patient and to provide mating cutting surfaces to that of the blade.

### BACKGROUND OF THE INVENTION

1. Field of the Invention

This application claims priority to U.S. Provisional Patent Application Ser. No. 61/909,294 filed on Nov. 26, 2013, and is inclusive thereof in its entirety by this reference thereto.

The disclosed device relates to endoscopic and arthrosporic and generally, surgical instruments. More particularly, the device herein disclosed and described relates to a surgical blade configured for engagement to a guide or cannula, and having an elongated aperture communicating through the blade rendering it flexible and providing a view through the blade for a cabled imaging device conventionally employed in surgery. The device is especially well adapted for employment during endoscopic surgery such as ligament release surgery and similar surgical procedures where the view for the surgeon is provided by a cabled imaging component such as a fiber optic engaged camera.

2. Prior Art

Many surgical procedures on patients for correction of an injury or a problem brought about by disease requires the surgeon to make incisions for removing, releasing or relocating body tissue. For instance, carpal tunnel syndrome is a common workplace related injury afflicting a significant percentage of the American workforce. Those afflicted experience numbness, tingling or burning sensations in the thumb and fingers. Those suffering may also experience wrist pain, pain in the hands, or loss of grip and strength. Often these symptoms prevent an employee from accomplishing routine tasks and inhibit activities in daily life.

For instance, carpal tunnel syndrome occurs when the median nerve is compressed at the wrist by the transverse carpal ligament. This increase in pressure may be caused by numerous genetic, environmental and physiological factors. The most common perceived cause of carpal tunnel syndrome is repetitive motion and computer users are at an elevated risk.

Surgical treatment of carpal tunnel syndrome is typical of other arthroscopic procedures and entails the surgical release of the transverse carpal ligament. The procedure consists of arthroscopically entering the body of the patient and using a cabled imaging device such as a fiber optic engaged camera, severing only the transverse carpal ligament with a anterograde knife, or retrograde knife or other blade as shown for instance in U.S. Pat. No. 5,651,790 made part hereof by this reference.

Herein lies a significant shortcoming in the prior art. Employing a cabled imaging component such as a fiber optic engaged camera and lighting system, visibility during the operation is minimal and somewhat unnatural. However, the need for surgical precision and the surgeon's complete confidence of the positioning accuracy of the cutting blade, is paramount.

A surgeon must be certain the desired flesh to yield the correct surgical outcome is being severed. Concurrently, the surgeon must be certain that surrounding nerves, ligaments, veins, arteries, and other internal organs are not damaged.

Typically, where a ligament is to be severed, it is positioned in the body of the patient adjacent blood vessels, nerves, and other ligaments. The surgeon employs a view of the procedure depicted on a video display, to ascertain the location of the blade, and concurrently to be certain cutting a chosen target on the display, will not damage a nerve or other adjacent body parts.

The environment in which such a surgery takes place is out of the direct sight of the surgeon who depends upon the video displayed image from the imaging device inserted at the surgical site. The view on the screen can be extremely crowded with multiple body structures appearing on the camera-provided view. Such can provide poor visibility to the surgeon, and concurrently fail to render many surrounding obstacles.

If the path leading to the site of a conventional ligament release or other arthroscopic procedure is blocked or contains obstructions such as nerves and blood vessels, using the blade devices of prior art, the patient may be harmed if the blade and blade guide are advanced in a linear approach and block the view of the camera of body parts behind but adjacent the blade. Further in some modes, a linear approach to the cutting site may not be the best approach. Therefor, a blade providing a view though the body of the blade would be of great value to the surgeon trying to discern what lies behind the blade on a video display. Further, a flexible blade capable of translation through the pathway of a curved guide component could also provide a significant advantage during a communication through the delicate interiors of the human body. Thus, a surgeon could view the surrounding body structures better and could reach otherwise obfuscated locations for surgery without disturbing and agitating surrounding tissue with a more flexible blade.

As such, there is a continuing unmet need for an improved surgical blade device which provides the incision and severing capabilities of a surgical blade, but is configured with a body providing increased visibility of internal organs and structures located on the opposite side of the blade from the viewing lens during endoscopic and arthroscopic procedures. Such a device if configured with a viewing aperture can also have significantly improved flexibility. Such would to allow the blade to be employed with conventional linear guides, or with curved blade guides to allow for flexibility in reaching a surgical site. Finally, such a surgical blade should be small in size and be available in single-use, disposable forms.

### SUMMARY OF THE INVENTION

The device and method of manufacture and employment herein disclosed and described achieves the above-mentioned goals, and provides a remedy for the noted shortcomings of prior art through the provision of a significantly enhanced surgical blade. This enhanced utility is achieved through the provision of a flexible surgical blade having a body configured with a viewing aperture communicating between the sides of the blade. This viewing aperture, during use with a cabled imaging component depicting a an image on a video display, significantly improves a surgeon's view of the internal surgical site when it is depicted on the video display from a lens adjacent the blade.

This improvement provided by the construction of the blade herein, provides the user a view both adjacent the facing side of the blade, and through the blade to view tissue on the other side from the camera component. Such a dual sided view allows the surgeon or user assurance that nerves, blood vessels and other parts of the body, located on the opposite side of the blade from the viewing component, are not being severed during a surgical procedure, since they can be viewed on the display through the aperture.

This view through the body of the blade thus provides a significant enhancement to the functionality of surgical blades employed for instance for internal, camera-viewed procedures such as endoscopic or arthroscopic ligament release surgeries and other surgical procedures. This viewing aperture formed in the body of the blade thus serves to increase cutting accuracy while preventing mistakes since the user can see and is assured that tissue behind the blade from the view of the camera lens is not being severed. A better view of the surgical site yields increased accuracy, preventing accidental severing of nerves and blood vessels and serves to increase the confidence of surgeons thereby leading to higher surgery success rates and faster operative times.

The blade device is preferably formed of metal but can also be formed of plastic or ceramic or similar materials. The blade itself has a handle engaging a body which is configured in a shape conducive to running within a track or pathway formed in a surgical guide used in combination herewith.

The body forming the blade formed with such material and having a large aperture formed within the side edges of the blade, also yields flexible characteristics to the blade itself. This desired blade flex property may be customized for different modes of the device depending upon the use of the blade. A narrow profile of the body of the blade approaching the distal end opposite the handle end, along with the lessened material forming the body due to the aperture, yields a blade which is sturdy in the plane of the blade, but flexible and capable of curving toward a plane traverse to that of the unbent mode of the blade.

This enhanced flexibility allows the blade device if desired, to flex and follow a curved pathway formed in a surgical guide. Such allows the blade to curve around obstacles thereby limiting damage and agitation of surrounding tissue when translated into compact and crowded internal cavities. The device's small nature and ability to navigate complex pathways, as well as being equipped with a viewing aperture can enable a single-portal incision for the surgical procedure where two entry points may have been needed with conventional devices.

In all modes of the device, the narrow profile of the body of the blade, and the aperture formed within the side edges thereof and providing a view for the camera through the blade, will help to decrease procedure times and a single-portal incision due to increased confidence of proper placement of the blade by the surgeon. Such of course can help minimize subsequent discomfort and recovery time for patients.

In all modes of the device, the novel viewing aperture allows the lens of an endoscopic camera to maintain a line of sight through the body of blade. Unlike conventional blades where this view is blocked by the facing or proximate side surface of the blade, the line of sight provided through the aperture of the disclosed blade device, significantly increases the remote internal visibility of tissues for the surgeon. The enhanced viewing in a crowded surgical site, provides the surgeon with enhanced confidence they are not severing desirable tissue and thereby will increase efficiency and lessen surgical duration.

For instance, in one use of the device herein for carpal tunnel surgery, allowing the surgeon to maintain visual contact with the median nerve prevents concern that using the blade when employed for cutting will result in complications from median nerve damage. The increased visibility also allows a surgeon to possess greater confidence in their accuracy allowing procedures to continue where using prior art blades with impaired viewing they might be terminated.

Different modes of the device may be configured with varied blade surfaces at the distal end or along the edge running within the pathway of the surgical guide. The device, preferably employed in combination with an appropriate blade guide, may be used as a push blade with the implementation of an anterograde blade, as a pull blade through the use of a retrograde-bladed distal end, or as a cutting blade through rotation of the blade at a rotation point, proximal to the handle. In all such modes, the provision of a viewing aperture enhances performance and utility to the surgeon or user. The device may also be engaged with a single use indicator thereby preventing the reuse of single use models and ensuring proper surgical hygiene.

The viewing aperture disclosed herein may be an opening in the blade communicating between both sides, or the aperture may surround with a transparent and flexible polymeric or plastic panel, to prevent tissue from becoming entangled within the device. However, currently the opening providing passage through the blade during use is preferred since it provides both a viewing passage as well as a passage for traverse of other surgical instruments therethrough if needed.

It is an object of this invention to provide a surgical blade which does not obstruct the view of body tissue on one side of the blade when viewed by a camera component from the opposite side of the blade.

It is an object of the device to provide a ligament release surgical device for the surgical treatments such as for carpal tunnel syndrome.

It is a further object of the device in this application to provide such a surgical device, which is flexible in the plane of the blade allowing for a curved path along a guide pathway to a surgical site which enables the surgeon to avoid surrounding tissues and nerves while performing a single-portal surgery thereby limiting internal trauma to the patient.

It is a further object of this device to provide such a surgical device which may also employ a transparent viewing aperture to increase visibility within the working area thereby mitigating the risk of median nerve complications.

It is yet another object to provide such a surgical blade which includes a viewing aperture whether configured as a push, pull, or rotatable cutting implement.

These together with other objects and advantages which become subsequently apparent reside in the details of the construction and operation as more fully hereinafter described and claimed, reference being had to the accompanying drawings forming a part thereof, wherein like numerals refer to like parts throughout.

With respect to the above summary, before explaining at least one preferred embodiment of the herein disclosed ligament release surgical tool in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

### BRIEF DESCRIPTION OF DRAWING FIGURES

FIG. 1 is an isometric perspective view of the disclosed blade device in which provides an aperture through the body for viewing therethrough.

FIG. 2 depicts another mode of the blade device translatably engaged in the pathway of a surgical guide and optionally adapted to rotate at the handle end of the body of the blade, for downward cutting between the blade and guide.

FIG. 2a is a side view of the blade and guide device translatably positioned within the guide which has an endwall for cutting against the distal end of the blade. The endwall might also be configured as a blade for a pull cutting.

FIG. 3 shows the disclosed blade device engaged as in FIG. 2a but with a perpendicular distal edge on both blade and guide.

FIG. 4 depicts the device shown engaged in the pathway of the guide device subsequent to a cutting of tissue. Also shown is a mode of the device which might project slightly from the distal end of the guide.

FIG. 5 depicts the blade device engaged in a curved pathway of a surgical guide showing the flexible nature of the blade device with aperture.

### DETAILED DESCRIPTION OF THE PREFERRED EMBODIMENTS OF THE INVENTION

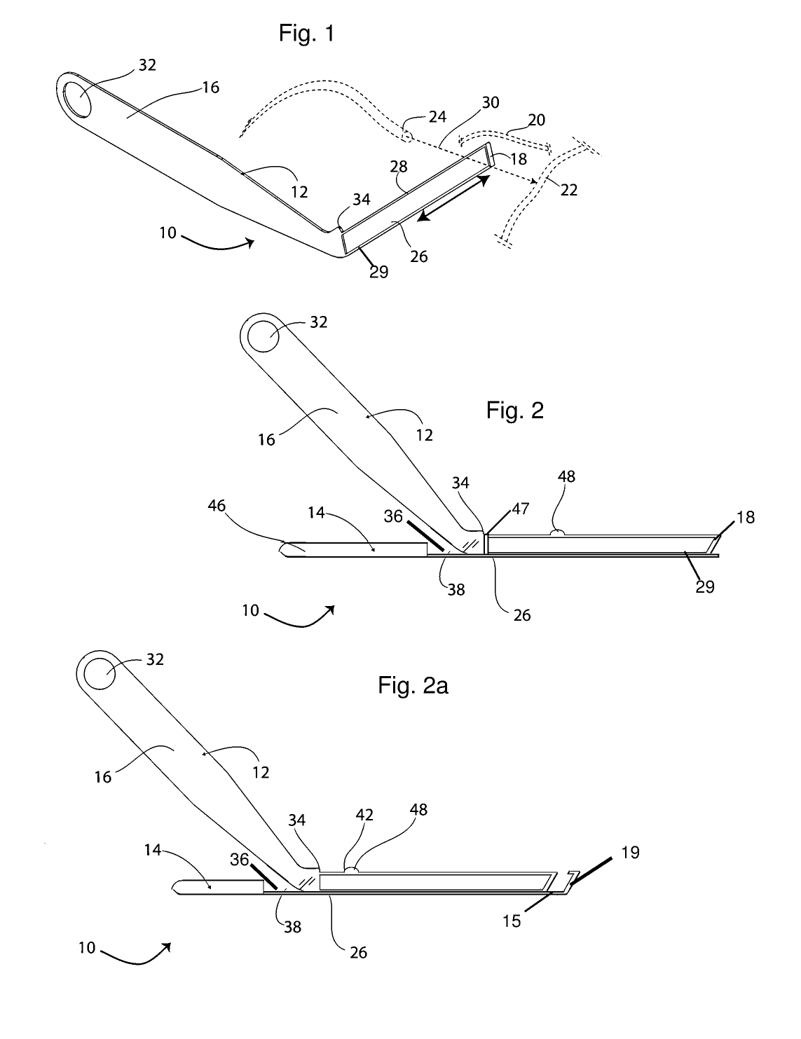

Referring now to the drawings of FIGS. 1-5, there is depicted a mode of the device 10 which depicts the aperture 26 communicating through the body forming the blade component 12 herein and which could be employed in any surgical blade instrument for increased utility. The description herein therefor should not be limiting as to the configuration or use of the blade component 12 as shown in the device 10 herein as it is anticipated the aperture 26 combined with any blade component 12 will provide a significant increase in utility to any surgical blade for any surgical purpose and such is anticipated.

As shown in FIG. 1, the device 10 includes the blade component 12 which is configured for severing the transverse carpal ligament during endoscopic ligament release surgery through the use of a blade component 12 and a guide component 14. This ligament severing is conducted through traditional means where a cabled imaging device provides a view therefrom on a display screen used by the surgeon to position the blade for cutting. With the added utility provided by the novel improvements mentioned herein, such surgery may be conducted with improved safety, visibility and accuracy, while reducing the risk of median nerve damage.

In FIG. 1, an isometric-perspective view of the device 10 is shown in an endoscopic ligament release embodiment without a complimentary guide component 14. The device 10 in the simplest mode employs a blade component 12 which may be employed with or without a guide component 14 which is conventionally formed of metal alloy or flexible plastic materials. The body forming the blade component 12, has a handle 16 extending from a proximal end.

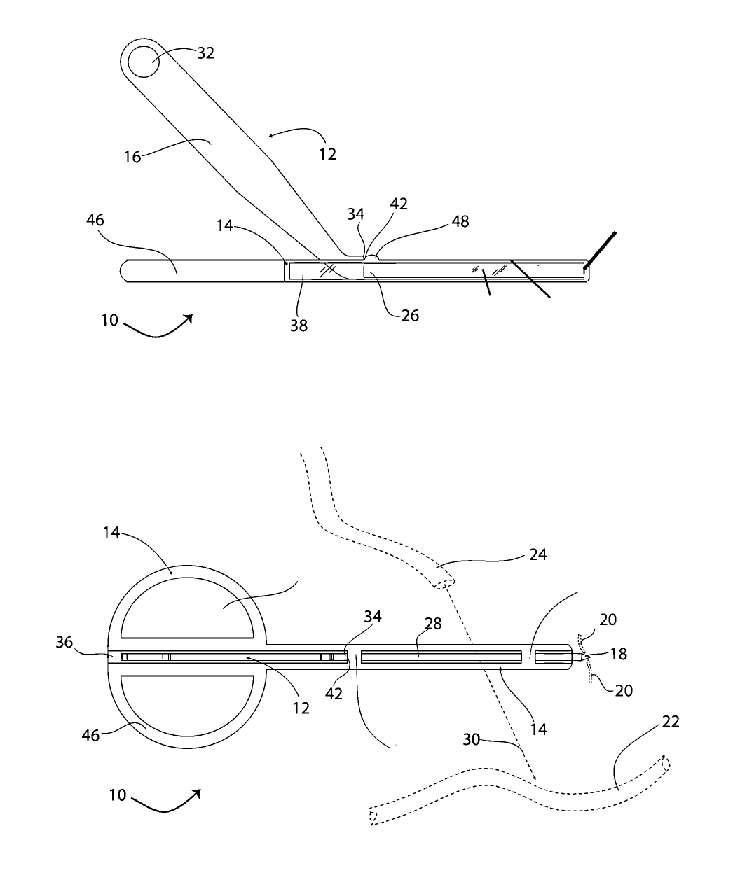

The distal end of the blade component 12 in the mode of FIG. 1, includes an anterograde blade 18 extending in a direction traverse to that of the lower edge 29 of the body 28 of the blade component. However as noted earlier, the blade component 12 may also be configured as a pull type blade, or as a rocking type blade as in FIGS. 2a and 3.

In use, the blade component 12 with the anterograde blade 18 engaged with a guide component 14, is inserted into a portal in the patient's body in the affected area and then carefully guided to the surgical site, through the use of the engaged guide component 14 which may also have a handle end.

The anterograde blade 18 shown in FIG. 1 and FIG. 2a is employable as a push-blade to cut an separate the transverse carpal ligament 20 and relieve the pressure on the median nerve 22.

When employed proximate to a blood vessel or a nerve such as the median nerve 22, the adjacent nerve or vessel must be protected from cutting or nicking during surgery and remain undamaged during the procedure. It is thus beneficial to maintain visual contact with the tissue, vessel, or nerve shown here as the median nerve 22, via the endoscopic video component 24. This endoscopic video component 24 communicates video information back to the surgeon for viewing on an electronic display, and allows the user to navigate the working area efficiently.

Due to the crowded and low visibility nature of the working area withing the confines of the body, the viewing passaged provided by the by a viewing aperture 26 in the planar member defining the body 28 of the blade component 12, allows the video component 24, to provide a view through the aperture 26 of the area of the body of the patient, on the opposite side of the blade component 12 from the video component 24. This viewing aperture 26 as noted may be a void, or if desirable, may be filled with a polymeric or plastic or similar transparent, material 27 such as shown in FIG. 3, and still provide the view of the opposite side of the blade component 12.

Using this view through the blade component 12 provided by the blade viewing aperture 26, the endoscopic video component 24 maintains a line of sight 30, through the body 28 of the blade component 12, with the tissue, vessel, or nerve, shown as the median nerve 22 on the opposite side of the blade component 12. This improved line of sight and increased visibility allows the surgeon to make a more confident cut through the targeted tissue such as the depicted transverse carpal ligament 20 while mitigating the risk of collateral median nerve 22 damage and tissue trauma.

Included as part of the device 10 in one mode, may be an attached use indicator void 32. This use indicator void 32 may engage with a plastic piece with a low temperature melting point, a color changing material when exposed to oxygen, or any other form or method to show a previous use, and prevent the reuse or re-sterilization of a single use embodiment of the device 10. As shown it indicates the device 10 has been used because the indicator is not present, and must be discarded.

Also included in a mode of the device 10 shown in FIGS. 1-5 is a stop, or stopping edge 34 which can be positioned to engage a stop 47 (FIG. 2) placed on the guide 14. This stopping edge 34 prevents the blade 12 from translating or protruding past the desired point along the pathway of the guide 14. It provides a safety measure to prevent unnecessary tissue damage to the surrounding anatomy beyond what is intended for cutting such as the shown ligament 20, when the blade component 12 is translated within the channel 36. The stopping edge 34 may also provide the same stopping characteristics when used with a complimentary cannula.

FIGS. 2-4 illustrate the device 10 where the blade 12 is employed with the aforementioned guide 14 configured in a linear or substantially straight mode. In modes where the top edge of the guide sidewall 15 rises above both sides and the aperture 26 formed in the blade component 12, such as FIG. 3, the guide 14 may have a guide viewing aperture 38 that aligns with the blade viewing aperture 26, to allow the endoscopic video component 24 to maintain its line of sight 30 with the median nerve 22. Transparent material may fill the guide viewing aperture 38 if desired.

Shown in FIGS. 2 and 3 is the blade 12 stowed within the channel 36 to prevent damage to surrounding tissue and anatomy during surgical insertion into the patient. As shown in FIG. 2, the stopping edge 34 prevents translation of the blade 12 past a stop 47 when positioned and the distal end of the guide 14 but allows a rocking of the blade 12 at the handle end, when a cutting edge is formed along the lower edge 29 the blade 12 which is positioned during insertion within the channel 36.

Once inserted into the patient and placed within the working area, the blade 12 may translate, or may rock or rotate relative the surface of the channel 36 in the guide 14, during the cutting procedure positioning tissue therebetween. In modes there the distal end of the blade 12 is employed for pushing and cutting, an anterograde blade edge 18 should be positioned within a vertical guide member 19 extending traverse to the channel 36 at the distal end of the guide 14 as shown in FIG. 2a or FIG. 3 until use.

When the anterograde blade edge 18 is fully exposed as in FIG. 2, a stopping edge 34 will contact a stop 47 positioned on the guide 14 as in FIG. 2 to prevent projection past the distal end of the guide 14. Projections 48 may also be placed on the blade 12 which will contact the appropriately positioned stop 47 as is shown in the drawings.

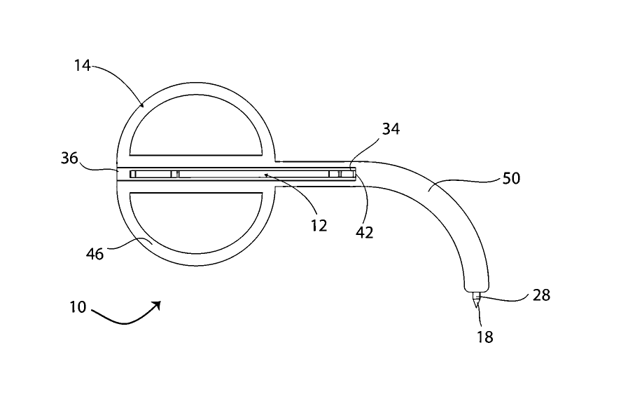

In FIG. 4 a top view of an exposed-blade mode where the cutting edge is on the distal end of the blade 12. Here as shown, the endoscopic component 24 can maintain a line of sight 30 with the nerve 22 through the aligned viewing aperture 26 of the blade and guide components. There is also present a mode of the guide handle 46 to allow securing of the guide component 14 to facilitate insertion into the working area and to prevent movement of the guide 14 during translational biasing acting on the blade 12.

Shown in FIGS. 4 and 5 is the channel 36 constrainment and tunnel constrainment of the blade 12 in the device 10. In FIG. 4 the stopping surface 42 is formed into a containment band 49 which constrains the blade 12 vertically within the channel 36. Likewise in FIG. 5 the stopping surface 42 is formed into a containment tunnel 50 which accomplishes the same vertical constrainment with the added benefit of preventing anatomy and tissue from becoming trapped in the channel 36 during device 10 insertion and removal.

FIG. 5 also illustrates a curved mode of the device 10. In this embodiment the guide 14 is formed as a curved member wherein the flexible blade 12 with aperture 26 therein, as disclosed herein, may follow the axial path of the guide 14 through the formed curved channel 36. This path curvature allows the device 10 to reach obfuscated internal regions of patients while reducing surrounding tissue agitation and trauma. Still present, but unseen in this figure are the viewing apertures of the blade 12 and guide 14 in any configuration herein.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. A surgical blade for cutting tissue, comprising:
a blade having a handle end and a distal edge;
said blade having opposing side edges communicating between said handle end and said distal edge;
an aperture formed in said blade between said opposing side edges, said handle end and said distal end; and
said aperture defining a line of sight for an imaging device adapted for use herewith and situated on a first side of said blade, said line of site extending from said imaging device, through said aperture, to an area adjacent a second side of said blade, opposite said first side.

2. The surgical blade of claim 1, additionally comprising:
a guide, said guide having a channel therein extending between a proximal end and a distal end of said guide;
a first side edge of said opposing side edges of said blade configured as a cutting edge for said blade;
said first side edge positionable within said channel to a protected positioning, with said channel surrounding said cutting edge, during an insertion of said blade into the body of a patient to an as-used position therein; and
said first side edge maneuverable to cut tissue in an area between said first side and said channel by a translation or rocking of said of said blade while in said as-used position.

3. The surgical blade of claim 1, additionally comprising:
a guide, said guide having a channel therein extending between a proximal end of said guide and a distal end of said guide;
a first side edge of said opposing side edges configured for positioning within said channel in a protective engagement, during an insertion of said blade into the body of a patient to an as-used position therein;
said distal edge configured as a cutting edge of said blade;
said distal edge maneuverable for cutting tissue while in said as-used position, by a translation of said first side edge within said channel in a direction toward or away from said distal end of said guide.

4. The surgical blade of claim 3, additionally comprising:
a vertical member extending from a distal end of said guide member;
a second channel formed therein configured to surround said distal edge with said first side edge in said protected positioning; and
said distal edge maneuverable for cutting said tissue while in said as-used position, by a translation of said first side edge within said channel, toward said vertical member with said tissue positioned between said distal edge and said vertical member.

5. The surgical blade of claim 4, additionally comprising:
said channel formed in said guide following a curved path between said proximal end of said guide and said distal end of said guide; and
said blade formed of flexible material whereby said blade follows said curved path during translation of said first side edge of said opposing side edges within said channel.

6. The surgical blade of claim 1, additionally comprising:
a transparent material positioned within said aperture.

7. The surgical blade of claim 2, additionally comprising:
a transparent material positioned within said aperture.

8. The surgical blade of claim 3, additionally comprising:
a transparent material positioned within said aperture.

9. The surgical blade of claim 4, additionally comprising:
a transparent material positioned within said aperture.

10. The surgical blade of claim 2, additionally comprising:
a first stop component positioned on said blade;
a second stop component positioned on said guide; and
contact between said first stop component and said second stop component preventing further translation of said blade toward said distal end of said guide.

11. The surgical blade of claim 3, additionally comprising:
a first stop component positioned on said blade;
a second stop component positioned on said guide; and
contact between said first stop component and said second stop component preventing further translation of said blade toward said distal end of said guide.

12. The surgical blade of claim 4, additionally comprising:
a first stop component positioned on said blade;
a second stop component positioned on said guide; and
contact between said first stop component and said second stop component preventing further translation of said blade toward said distal end of said guide.

13. The surgical blade of claim 5, additionally comprising:
a first stop component positioned on said blade;
a second stop component positioned on said guide; and
contact between said first stop component and said second stop component preventing further translation of said blade toward said distal end of said guide.

14. The surgical blade of claim 7, additionally comprising:
a first stop component positioned on said blade;
a second stop component positioned on said guide; and
contact between said first stop component and said second stop component preventing further translation of said blade toward said distal end of said guide.

15. The surgical blade of claim 8, additionally comprising:
a first stop component positioned on said blade;
a second stop component positioned on said guide; and
contact between said first stop component and said second stop component preventing further translation of said blade toward said distal end of said guide.

16. The surgical blade of claim 9, additionally comprising:
a first stop component positioned on said blade;
a second stop component positioned on said guide; and
contact between said first stop component and said second stop component preventing further translation of said blade toward said distal end of said guide.
